(19) United States
(12) Patent Application Publication
(10) Pub. No.: US 2019/0167786 A1
(43) Pub. Date: Jun. 6, 2019

(54) EMULSIONS WITH FREE AQUEOUS-PHASE SURFACTANT FOR ADJUVANTING SPLIT INFLUENZA VACCINES

(71) Applicant: Seqirus UK Limited, Berkshire (GB)

(72) Inventor: Derek O'Hagan, Berkeley, CA (US)

(21) Appl. No.: 16/029,863

(22) Filed: Jul. 9, 2018

Related U.S. Application Data
(63) Continuation of application No. 12/092,131, filed on Dec. 15, 2008, now abandoned.
(60) Provisional application No. 60/734,026, filed on Nov. 4, 2005.
(60) Provisional application No. 60/812,476, filed on Jun. 8, 2006.

(51) Int. Cl. A61K 39/39; A61K 39/12; A61K 39/145

(52) U.S. Cl. CPC A61K 39/39; A61K 2039/55572; A61K 39/145; A61K 39/12

(57) ABSTRACT

A split influenza virus vaccine is adjuvanted with an oil-in-water emulsion that contains free surfactant in its aqueous phase. The free surfactant can continue to exert a ‘splitting effect’ on the antigen, thereby disrupting any unsplit virions and/or virion aggregates that might be present.

### CROSS-REFERENCE TO RELATED APPLICATIONS

This application is a continuation of U.S. patent application Ser. No. 12/092,131, filed Dec. 15, 2008, which is the United States national stage entry under 35 U.S.C. § 371 of International Application No. PCT/GB2006/004139, filed Nov. 6, 2006, which claims the benefit of U.S. Provisional Application No. 60/734,026, filed Nov. 4, 2005 and U.S. Provisional Application No. 60/812,476, filed Jun. 8, 2006. The contents of these applications are each incorporated herein by reference in their entirety.

All documents cited herein are incorporated by reference in their entirety.

### TECHNICAL FIELD

This invention is in the field of vaccines for protecting against influenza virus infection, and in particular split vaccines.

### BACKGROUND ART

Influenza vaccines are described in chapters 17 & 18 of reference 1. They are based on live virus or inactivated virus, and inactivated vaccines can be based on whole virus, ‘split’ virus or on purified surface antigens (including hemagglutinin and neuraminidase). Haemagglutinin (HA) is the main immunogen in inactivated influenza vaccines, and vaccine doses are standardized by reference to HA levels, with vaccines typically containing about 15 μg of HA per strain.

The ‘split’ vaccines are obtained by treating virions with detergents to produce subvirion preparations, using methods such as the ‘Tween-ether’ splitting process. Split vaccines generally include multiple antigens from the influenza virion. The BEGRIVAC™, FLUARIX™, FLUZONE™ and FLUSHIELD™ products are split vaccines.

During the 2000-01 season in Canada, a newly-identified oculorespiratory syndrome (ORS) was observed in patients who received split vaccines. The ORS has been associated with incomplete splitting of virions during manufacture, giving compositions with a high proportion of microaggregates of unsplit virions [2].

There is no causal explanation of the link between split vaccines and ORS, but the clinical and epidemiological features of ORS are suggestive of hypersensitivity, and so it has been proposed that the vaccine may upset the natural Th1/Th2 balance, with the particulate unsplit virions causing a bias towards a Th2 phenotype. In reference 3, for example, the presence of aggregates in split influenza vaccines was found to deviate the immune response to a greater Th2 cytokine pattern. In reference 4, however, no link could be confirmed between ORS and the Th1/Th2 balance.

In a situation where influenza vaccines have to be produced in a hurry (e.g. in a pandemic outbreak) then pressures on manufacturers might inadvertently result in the release of vaccines that suffer from the same problems as the partially-unsplit aggregated Canadian batches from 2000-01. Indeed, reference 2 states that “it may not be possible to eliminate unsplit virions and aggregates altogether”, and that “some low-level risk for triggering ocular and respiratory symptoms may be unavoidable”.

It is an object of the invention to minimize the risk that a split influenza vaccine might suffer from the same problems as those seen in Canada in the 2000-01 season.

### DISCLOSURE OF THE INVENTION

The invention meets this object by adjuvanting a split influenza virus vaccine with an oil-in-water emulsion that contains free surfactant in its aqueous phase. The free surfactant can continue to exert a ‘splitting effect’ on the antigen, thereby disrupting any unsplit virions and/or virion aggregates that might otherwise be present. Moreover, although free surfactant might be expected over time to have a denaturing effect on membrane glycoproteins, such as the important HA antigen, the short shelf-life required for a typical influenza vaccine means that this issue should not cause difficulties in practice.

Thus the invention provides an immunogenic composition comprising a split influenza virus antigen and an oil-in-water emulsion, wherein the emulsion includes free surfactant in its aqueous phase.

The invention also provides a method for preparing an immunogenic composition comprising the steps of combining: (i) a split influenza virus antigen; and (ii) an oil-in-water emulsion that includes free surfactant in its aqueous phase.

The invention also provides a kit comprising: (i) a first kit component comprising a split influenza virus antigen; and (ii) a second kit component comprising an oil-in-water emulsion that includes free surfactant in its aqueous phase.

Although there are currently no adjuvanted split influenza vaccines on the market, there are several proposals for introducing adjuvants into influenza vaccines in order to permit an increased number of doses to be produced from a fixed amount of antigen. For example, references 5 to 8 disclose the use of aluminum salts to adjuvant whole virion influenza vaccines. The invention avoids the use of aluminum salts as the sole adjuvant for split vaccines because they promote a Th2-type immune response when used on their own, which was implicated in the Canadian ORS outbreak (see above).

The Split Influenza Virus Antigen

Compositions of the invention include an antigen obtained by splitting influenza virions. The split virion will typically include multiple antigens from the influenza virion, including hemagglutinin, neuraminidase, matrix and nucleoprotein. The invention does not encompass live virus vaccines (such as the FLUMIST™ product), whole-virion inactivated vaccines (such as the INFLEXAL™ product), purified surface antigen vaccines (which are based on purified hemagglutinin and neuraminidase surface glycoproteins, such as the FLUVIRIN™, AGRIPPAL™ and INFLUVAC™ products) or virosomal vaccines (which take the form of nucleic acid free viral-like liposomal particles [9], as in the INFLEXAL V™ and INVAVAC™ products).

Virions can be harvested from virus-containing fluids by various methods. For example, a purification process may involve zonal centrifugation using a linear sucrose gradient solution that includes detergent to disrupt the virions.

Split virions can be obtained by treating purified virions with detergents (e.g. ethyl ether, polysorbate 80, deoxycholate, tri-N-butyl phosphate, Triton X-100, Triton N101, cetyltrimethylammonium bromide, Tergitol NP9, etc.) to produce subvirion preparations, including the ‘Tween-ether’ splitting process. Methods of splitting influenza viruses are well known in the art e.g. see refs. 10−¹, etc. Splitting of the virus is typically carried out by disrupting or fragmenting whole virus, whether infectious or non-infectious with a disrupting concentration of a splitting agent. The disruption results in a full or partial solubilisation of the virus proteins, altering the integrity of the virus. Preferred splitting agents are non-ionic and ionic (e.g. cationic) surfactants e.g. alkylglycosides, alkylthioglycosides, acyl sugars, sulphobetaines, betains, polyoxyethylenealkylethers, N,N-dialkyl-Glucamides, Hecameg, alkylphenoxy-polyethoxyethanols, quaternary ammonium compounds, sarcosyl, CTABs (cetyl trimethyl ammonium bromides), tri-N-butyl phosphate, Cetavlon, myristyltrimethylammonium salts, lipofectin, lipofectamine, and DOT-MA, the octyl- or nonyl-phenoxy polyoxyethanols (e.g. the Triton surfactants, such as Triton X-100 or Triton N101), polyoxyethylene sorbitan esters (the Tween surfactants), polyoxyethylene ethers, polyoxyethlene esters, etc. One useful splitting procedure uses the consecutive effects of sodium deoxycholate and formaldehyde, and splitting can take place during initial virion purification (e.g. in a sucrose density gradient solution). Split virions can usefully be resuspended in sodium phosphate-buffered isotonic sodium chloride solution.

The influenza virus may be attenuated. The influenza virus may be temperature-sensitive. The influenza virus may be cold-adapted.

Influenza virus strains used in vaccines change from season to season. In the current inter-pandemic period, trivalent vaccines are typical, including two influenza A strains (H1N1 and H3N2) and one influenza B strain. The invention can be used with inter-pandemic strains of this type, but can also be used with viruses from pandemic strains (i.e. strains to which the vaccine recipient and the general human population are immunologically naïve), such as H2, H5, H7 or H9 subtype strains (in particular of influenza A virus), and influenza vaccines for pandemic strains may be monovalent or may, for instance, be based on a normal trivalent vaccine supplemented by a pandemic strain. Depending on the season and on the nature of the antigen included in the vaccine, however, the invention may protect against one or more of influenza A virus HA subtypes H1, H2, H3, H4, H5, H6, H7, H8, H9, H10, H11, H12, H13, H14, H15 or H16. The invention may protect against one or more of influenza A virus NA subtypes N1, N2, N3, N4, N5, N6. N7, N8 or N9.

As well as being suitable for immunizing against inter-pandemic strains, the compositions of the invention are particularly useful for immunizing against pandemic strains. The characteristics of an influenza strain that give it the potential to cause a pandemic outbreak are: (a) it contains a new HA compared to the HAs in currently-circulating human strains, i.e. one that has not been evident in the human population for over a decade (e.g. H2), or has not previously been seen at all in the human population (e.g. H5, H6 or H9, that have generally been found only in bird populations), such that the human population will be immunologically naïve to the strain's HA; (b) it is capable of being transmitted horizontally in the human population; and (c) it is pathogenic to humans. A virus with H5 haemagglutinin type is preferred for immunising against pandemic influenza, such as a H5N1 strain. Other possible strains include H5N3. H9N2, H2N2, H7N1 and H7N7, and any other emerging potentially pandemic strains. Within the H5 subtype, a virus may fall into HA clade 1, HA clade 1′, HA clade 2 or HA clade 3 [15], with clades 1 and 3 being particularly relevant.

Influenza virus strains used with the invention may be resistant to antiviral therapy (e.g. resistant to oseltamivir [16] and/or zanamivir), including resistant pandemic strains [17].

Compositions of the invention may include antigen(s) from one or more (e.g. 1, 2, 3, 4 or more) influenza virus strains, including influenza A virus and/or influenza B virus. Where a vaccine includes more than one strain of influenza, the different strains are typically grown separately and are mixed after the viruses have been harvested and split. Thus a process of the invention may include the step of mixing antigens from more than one influenza strain. A trivalent vaccine is preferred, including two influenza A virus strains and one influenza B virus strain.

In some embodiments of the invention, the compositions may include antigen from a single influenza A strain. In some embodiments, the compositions may include antigen from two influenza A strains, provided that these two strains are not H1N1 and H3N2. In some embodiments, the compositions may include antigen from more than two influenza A strains.

The influenza virus may be a reassortant strain, and may have been obtained by reverse genetics techniques. Reverse genetics techniques [e.g. 18-22] allow influenza viruses with desired genome segments to be prepared in vitro using plasmids. Typically, they involve expressing (a) DNA molecules that encode desired viral RNA molecules e.g. from poll promoters, and (b) DNA molecules that encode viral proteins e.g. from polII promoters, such that expression of both types of DNA in a cell leads to assembly of a complete intact infectious virion. The DNA preferably provides all of the viral RNA and proteins, but it is also possible to use a helper virus to provide some of the RNA and proteins. Plasmid-based methods using separate plasmids for producing each viral RNA are preferred 123-251, and these methods will also involve the use of plasmids to express all or some (e.g. just the PB1, PB2, PA and NP proteins) of the viral proteins, with 12 plasmids being used in some methods.

To reduce the number of plasmids needed, a recent approach [26] combines a plurality of RNA polymerase I transcription cassettes (for viral RNA synthesis) on the same plasmid (e.g. sequences encoding 1, 2, 3, 4, 5, 6, 7 or all 8 influenza A vRNA segments), and a plurality of protein-coding regions with RNA polymerase II promoters on another plasmid (e.g. sequences encoding 1, 2, 3, 4, 5, 6, 7 or all 8 influenza A mRNA transcripts). Preferred aspects of the reference 26 method involve: (a) PB1, PB2 and PA mRNA-encoding regions on a single plasmid; and (b) all 8 vRNA-encoding segments on a single plasmid. Including the NA and HA segments on one plasmid and the six other segments on another plasmid can also facilitate matters.

As an alternative to using poll promoters to encode the viral RNA segments, it is possible to use bacteriophage polymerase promoters [27]. For instance, promoters for the SP6, T3 or T7 polymerases can conveniently be used. Because of the species-specificity of poll promoters, bacteriophage polymerase promoters can be more convenient for many cell types (e.g. MDCK), although a cell must also be transfected with a plasmid encoding the exogenous polymerase enzyme.

In other techniques it is possible to use dual poll and polII promoters to simultaneously code for the viral RNAs and for expressible mRNAs from a single template [28,29].

Thus an influenza A virus may include one or more RNA segments from a A/PR/8/34 virus (typically 6 segments from A/PR/8/34, with the HA and N segments being from a vaccine strain, i.e. a 6:2 reassortant), particularly when viruses are grown in eggs. It may also include one or more RNA segments from a A/WSN/33 virus, or from any other virus strain useful for generating reassortant viruses for vaccine preparation. Typically, the invention protects against a strain that is capable of human-to-human transmission, and so the strain's genome will usually include at least one RNA segment that originated in a mammalian (e.g. in a human) influenza virus. It may include NS segment that originated in an avian influenza virus.

The viruses used as the source of the antigens can be grown either on eggs or on cell culture. The current standard method for influenza virus growth uses specific pathogen-free (SPF) embryonated hen eggs, with virus being purified from the egg contents (allantoic fluid). More recently, however, viruses have been grown in animal cell culture and, for reasons of speed and patient allergies, this growth method is preferred. If egg-based viral growth is used then one or more amino acids may be introduced into the allantoid fluid of the egg together with the virus [1].

When cell culture is used, the viral growth substrate will typically be a cell line of mammalian origin. Suitable mammalian cells of origin include, but are not limited to, hamster, cattle, primate (including humans and monkeys) and dog cells. Various cell types may be used, such as kidney cells, fibroblasts, retinal cells, lung cells, etc. Examples of suitable hamster cells are the cell lines having the names BHK21 or HKCC. Suitable monkey cells are e.g. African green monkey cells, such as kidney cells as in the Vero cell line. Suitable dog cells are e.g. kidney cells, as in the MDCK cell line. Thus suitable cell lines include, but are not limited to: MDCK; CHO; 293T; BHK; Vero; MRC-5; PER.C6; WI-38; etc. Preferred mammalian cell lines for growing influenza viruses include: MDCK cells [30-33], derived from Madin Darby canine kidney: Vero cells [34-36], derived from African green monkey (Cercopithecus aethiops) kidney; or PER.C6 cells [37], derived from human embryonic retinoblasts. These cell lines are widely available e.g. from the American Type Cell Culture (ATCC) collection [38], from the Coriell Cell Repositories [39], or from the European Collection of Cell Cultures (ECACC). For example, the ATCC supplies various different Vero cells under catalog numbers CCL-81, CCL-81.2, CRL-1586 and CRL-1587, and it supplies MDCK cells under catalog number CCL-34. PER.C6 is available from the ECACC under deposit number 96022940. As a less-preferred alternative to mammalian cell lines, virus can be grown on avian cell lines [e.g. refs. 40-42], including cell lines derived from ducks (e.g. duck retina) or hens e.g. chicken embryo fibroblasts (CEF), etc. Examples include avian embryonic stem cells [40,43], including the EBx cell line derived from chicken embryonic stem cells. EB45, EB14, and EB14-074 [44].

The most preferred cell lines for growing influenza viruses are MDCK cell lines. The original MDCK cell line is available from the ATCC as CCL-34, but derivatives of this cell line may also be used. For instance, reference 30 discloses a MDCK cell line that was adapted for growth in suspension culture (‘MDCK 33016’, deposited as DSM ACC 2219). Similarly, reference 45 discloses a MDCK-derived cell line that grows in suspension in serum-free culture (‘B-702’, deposited as FERM BP-7449). Reference 46 discloses non-tumorigenic MDCK cells, including ‘MDCK-S’ (ATCC PTA-6500), ‘MDCK-SF101’ (ATCC PTA-6501), ‘MDCK-SF102’ (ATCC PTA-6502) and ‘MDCK-SF103’ (PTA-6503). Reference 47 discloses MDCK cell lines with high susceptibility to infection, including ‘MDCK.5F1’ cells (ATCC CRL-12042). Any of these MDCK cell lines can be used.

For growth on a cell line, such as on MDCK cells, virus may be grown on cells in suspension [30,48,49] or in adherent culture. One suitable MDCK cell line for suspension culture is MDCK 33016 (deposited as DSM ACC 2219). As an alternative, microcarrier culture can be used.

Cell lines supporting influenza virus replication are preferably grown in serum-free culture media and/or protein free media. A medium is referred to as a serum-free medium in the context of the present invention in which there are no additives from serum of human or animal origin. Protein-free is understood to mean cultures in which multiplication of the cells occurs with exclusion of proteins, growth factors, other protein additives and non-serum proteins, but can optionally include proteins such as trypsin or other proteases that may be necessary for viral growth. The cells growing in such cultures naturally contain proteins themselves.

Cell lines supporting influenza virus replication are preferably grown below 37° C. [50] (e.g. 30-36° C., or at about 30° C., 31° C., 32° C., 33° C., 34° C., 35° C., 36° C.), for example during viral replication.

Where virus is grown on a cell line then the growth culture, and also the viral inoculum used to start the culture, is preferably free from (i.e. will have been tested for and given a negative result for contamination by) herpes simplex virus, respiratory syncytial virus, parainfluenza virus 3, SARS coronavirus, adenovirus, rhinovirus, reoviruses, polyomaviruses, birnaviruses, circoviruses, and/or parvoviruses [51]. Absence of herpes simplex viruses is particularly preferred.

Where virus has been grown on a mammalian cell line then the composition will advantageously be free from egg proteins (e.g. ovalbumin and ovomucoid) and from chicken DNA, thereby reducing allergenicity. The avoidance of allergens is a further way of minimizing Th2 responses.

Where virus has been grown on a cell line then the composition preferably contains less than 10 ng (preferably less than 1 ng, and more preferably less than 100 μg) of residual host cell DNA per dose, although trace amounts of host cell DNA may be present. In general, the host cell DNA that it is desirable to exclude from compositions of the invention is DNA that is longer than 100 bp.

Measurement of residual host cell DNA is now a routine regulatory requirement for biologicals and is within the normal capabilities of the skilled person. The assay used to measure DNA will typically be a validated assay [52,53]. The performance characteristics of a validated assay can be described in mathematical and quantifiable terms, and its possible sources of error will have been identified. The assay will generally have been tested for characteristics such as accuracy, precision, specificity. Once an assay has been calibrated (e.g. against known standard quantities of host cell DNA) and tested then quantitative DNA measurements can be routinely performed. Three principle techniques for DNA quantification can be used: hybridization methods, such as Southern blots or slot blots [54]; immunoassay methods, such as the Threshold™ System [55]; and quantitative PCR [56]. These methods are all familiar to the skilled person, although the precise characteristics of each method may depend on the host cell in question e.g. the choice of probes for hybridization, the choice of primers and/or probes for amplification, etc. The Threshold™ system from Molecular Devices is a quantitative assay for picogram levels of total DNA, and has been used for monitoring levels of contaminating DNA in biopharmaceuticals [55]. A typical assay involves non-sequence-specific formation of a reaction complex between a biotinylated ssDNA binding protein, a urease-conjugated anti-ssDNA antibody, and DNA. All assay components are included in the complete Total DNA Assay Kit available from the manufacturer. Various commercial manufacturers offer quantitative PCR assays for detecting residual host cell DNA e.g. AppTec™ Laboratory Services, BioReliance™, Althea Technologies, etc. A comparison of a chemiluminescent hybridisation assay and the total DNA Threshold™ system for measuring host cell DNA contamination of a human viral vaccine can be found in reference 57.

Contaminating DNA can be removed during vaccine preparation using standard purification procedures e.g. chromatography, etc. Removal of residual host cell DNA can be enhanced by nuclease treatment e.g. by using a DNase. A convenient method for reducing host cell DNA contamination is disclosed in references 58 & 59, involving a two-step treatment, first using a DNase (e.g. Benzonase), which may be used during viral growth, and then a cationic detergent (e.g. CTAB), which may be used during virion disruption. Treatment with an alkylating agent, such as j-propiolactone, can also be used to remove host cell DNA, and advantageously may also be used to inactivate virions [60].

Vaccines containing <10 ng (e.g. <1 ng, <100 μg) host cell DNA per 15 μg of haemagglutinin are preferred, as are vaccines containing <10 ng (e.g. <1 ng, <100 μg) host cell DNA per 0.25 ml volume. Vaccines containing <10 ng (e.g. <1 ng, <100 μg) host cell DNA per 50 μg of haemagglutinin are more preferred, as are vaccines containing <10 ng (e.g. <1 ng, <100 μg) host cell DNA per 0.5 ml volume.

The method for propagating virus in cultured cells generally includes the steps of inoculating the cultured cells with the strain to be cultured, cultivating the infected cells for a desired time period for virus propagation, such as for example as determined by virus titer or antigen expression (e.g. between 24 and 168 hours after inoculation) and collecting the propagated virus. The cultured cells are inoculated with a virus (measured by PFU or TCID₅₀) to cell ratio of 1:500 to 1:1, preferably 1:100 to 1:5, more preferably 1:50 to 1:10. The virus is added to a suspension of the cells or is applied to a monolayer of the cells, and the virus is absorbed on the cells for at least 60 minutes but usually less than 300 minutes, preferably between 90 and 240 minutes at 25° C. to 40° C., preferably 28° C. to 37° C. The infected cell culture (e.g. monolayers) may be removed either by freeze-thawing or by enzymatic action to increase the viral content of the harvested culture supernatants. The harvested fluids are then either inactivated or stored frozen. Cultured cells may be infected at a multiplicity of infection (“m.o.i.”) of about 0.0001 to 10, preferably 0.002 to 5, more preferably to 0.001 to 2. Still more preferably, the cells are infected at a m.o.i of about 0.01. Infected cells may be harvested 30 to 60 hours post infection. Preferably, the cells are harvested 34 to 48 hours post infection. Still more preferably, the cells are harvested 38 to 40 hours post infection. Proteases (typically trypsin) are generally added during cell culture to allow viral release, and the proteases can be added at any suitable stage during the culture.

Haemagglutinin (HA) is the main immunogen in inactivated influenza vaccines, including in split vaccines, and vaccine doses are standardised by reference to HA levels, typically as measured by a single radial immunodiffusion (SRID) assay. Existing split vaccines typically contain about 15 μg of HA per strain, although lower doses are also used e.g. for children, or in pandemic situations. Fractional doses such as ½ (i.e. 7.5 μg HA per strain), ‘ and’/s have been used [7.8], as have higher doses (e.g. 3× or 9× doses [61,62]). Thus vaccines may include between 0.1 and 150 μg of HA per influenza strain, preferably between 0.1 and 50 μg e.g. 0.1-20 μg, 0.1-15 μg, 0.1-10 μg, 0.1-7.5 μg, 0.5-5 μg, etc. Particular doses include e.g. about 45, about 30, about 15, about 10, about 7.5, about 5, about 3.8, about 1.9, about 1.5, etc. per strain. The inclusion of an adjuvant in the vaccine can compensate for the lower inherent immunogenicity of these lower doses.

HA used with the invention may be a natural HA as found in a virus, or may have been modified. For instance, it is known to modify HA to remove determinants (e.g. hyper-basic regions around the cleavage site between HA1 and HA2) that cause a virus to be highly pathogenic in avian species, as these determinants can otherwise prevent a virus from being grown in eggs.

Compositions of the invention may include detergent e.g. a polyoxyethylene sorbitan ester surfactant (known as ‘Tweens’), an octoxynol (such as octoxynol-9 (Triton X-100) or t-octylphenoxypolyethoxyethanol), a cetyl trimethyl ammonium bromide (‘CTAB’), or sodium deoxycholate, particularly for a split or surface antigen vaccine. The detergent may be present only at trace amounts. Thus the vaccine may included less than 1 mg/ml of each of octoxynol-10, α-tocopheryl hydrogen succinate and polysorbate 80. Other residual components in trace amounts could be antibiotics (e.g. neomycin, kanamycin, polymyxin B).

The Oil-in-Water Emulsion

Oil-in-water emulsions have been found to be particularly suitable for use in adjuvanting influenza virus vaccines. Various such emulsions are known, and they typically include at least one oil and at least one surfactant, with the oil(s) and surfactant(s) being biodegradable (metabolisable) and biocompatible. The oil droplets in the emulsion are generally less than 5 μm in diameter, and may even have a sub-micron diameter, with these small sizes being achieved with a microfluidiser to provide stable emulsions. Droplets with a size less than 220 nm are preferred as they can be subjected to filter sterilization.

The invention can be used with oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoids known as squalene, 2,6,10,15,19,23-hexamethyl-2,6,10,14,18,22-tetracosahexaene, which is particularly preferred herein. Squalane, the saturated analog to squalene, is also a preferred oil. Fish oils, including squalene and squalane, are readily available from commercial sources or may be obtained by methods known in the art. Other preferred oils are the tocopherols (see below). Mixtures of oils can be used.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethox (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); nonylphenol ethoxylates, such as the Tergitol™ NP series; polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Non-ionic surfactants are preferred. Preferred surfactants for including in the emulsion are Tween 80 (polyoxyethylene sorbitan monooleate), Span 85 (sorbitan trioleate), lecithin and Triton X-100.

Mixtures of surfactants can be used e.g. Tween 80/Span 85 mixtures. A combination of a polyoxyethylene sorbitan ester such as polyoxyethylene sorbitan monooleate (Tween 80) and an octoxynol such as t-octylphenoxypolyethoxyethanol (Triton X-100) is also suitable. Another useful combination comprises laureth 9 plus a polyoxyethylene sorbitan ester and/or an octoxynol.

Preferred amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Whatever the choice of oil(s) and surfactant(s), the surfactant(s) is/are included in excess of the amount required for emulsification, such that free surfactant remains in the aqueous phase. Free surfactant in the final emulsion can be detected by various assays. For instance, a sucrose gradient centrifugation method can be used to separate emulsion droplets from the aqueous phase, and the aqueous phase can then be analyzed. Centrifugation can be used to separate the two phases, with the oil droplets coalescing and rising to the surface, after which the surfactant content of the aqueous phase can be determined e.g. using HPLC or any other suitable analytical technique.

Specific oil-in-water emulsion adjuvants useful with the invention include, but are not limited to:
A submicron emulsion of squalene, Tween 80, and Span 85. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ [63-65], as described in more detail in Chapter 10 of ref. 66 and chapter 12 of ref. 67. The MF59 emulsion advantageously includes citrate ions e.g. 10 mM sodium citrate buffer.
An emulsion of squalene, a tocopherol, and Tween 80. The emulsion may include phosphate buffered saline. It may also include Span 85 (e.g. at 1%) and/or lecithin. These emulsions may have from 2 to 10% squalene, from 2 to 10% tocopherol and from 0.3 to 3% Tween 80, and the weight ratio of squalene:tocopherol is preferably <1 as this provides a more stable emulsion. Squalene and Tween 80 may be present volume ratio of about 5:2. One such emulsion can be made by dissolving Tween 80 in PBS to give a 2% solution, then mixing 90 ml of this solution with a mixture of (5 g of DL-α-tocopherol and 5 ml squalene), then microfluidising the mixture. The resulting emulsion may have submicron oil droplets e.g. with an average diameter of between 100 and 250 nm, preferably about 180 nm.
An emulsion of squalene, a tocopherol, and a Triton detergent (e.g. Triton X-100). The emulsion may also include a 3d-MPL (see below). The emulsion may contain a phosphate buffer.
An emulsion comprising a polysorbate (e.g. polysorbate 80), a Triton detergent (e.g. Triton X-100) and a tocopherol (e.g. an α-tocopherol succinate). The emulsion may include these three components at a mass ratio of about 75:11:10 (e.g. 750 g/ml polysorbate 80, 110 g/ml Triton X-100 and 100 μg/ml α-tocopherol succinate), and these concentrations should include any contribution of these components from antigens. The emulsion may also include squalene. The emulsion may also include a 3d-MPL (see below). The aqueous phase may contain a phosphate buffer.
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant [681 (0.05-1% Thr-MDP, 5% squalane, 2.5% Pluronic L121 and 0.2% polysorbate 80). It can also be used without the Thr-MDP, as in the “AF” adjuvant 169] (5% squalane, 1.25% Pluronic L121 and 0.2% polysorbate 80). Microfluidisation is preferred.
An emulsion having from 0.5-50% of an oil, 0.1-10% of a phospholipid, and 0.05-5% of a non-ionic surfactant. As described in reference 70, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in reference 71, produced by addition of aliphatic amine to desacylsaponin via the carboxyl group of glucuronic acid), dimethyidioctadecylammonium bromide and/or N,N-dioctadecyl-N,N-bis (2-hydroxyethyl)propanediamine.
An emulsion in which a saponin (e.g. QuilA or QS21) and a sterol (e.g. a cholesterol) are associated as helical micelles [72].

The emulsions and split antigen may be mixed during manufacture, before packaging, or they may be mixed extemporaneously, at the time of delivery. Thus the adjuvant and antigen may be kept separately in a packaged or distributed vaccine, ready for final formulation at the time of use. The antigen will generally be in an aqueous form, such that the vaccine is finally prepared by mixing two liquids. The volume ratio of the two liquids for mixing can vary (e.g. between 5:1 and 1:5) but is generally about 1:1. Suitable kits are described in more detail below.

After the antigen and adjuvant have been mixed, haemagglutinin antigen will generally remain in aqueous solution but may distribute itself around the oil/water interface. In general, little if any haemagglutinin will enter the oil phase of the emulsion.

Where a composition includes a tocopherol, any of the α, β, γ, δ, ε or ξ tocopherols can be used, but α-tocopherols are preferred. The tocopherol can take several forms e.g. different salts and/or isomers. Salts include organic salts, such as succinate, acetate, nicotinate, etc. D-α-tocopherol and DL-α-tocopherol can both be used. Tocopherols are advantageously included in vaccines for use in elderly patients (e.g. aged 60 years or older) because vitamin E has been reported to have a positive effect on the immune response in this patient group [73]. They also have antioxidant properties that may help to stabilize the emulsions [74]. A preferred α-tocopherol is DL-α-tocopherol, and the preferred salt of this tocopherol is the succinate. The succinate salt has been found to cooperate with TNF-related ligands in vivo. Moreover, α-tocopherol succinate is known to be compatible with influenza vaccines and to be a useful preservative as an alternative to mercurial compounds [14]. In addition, vitamin E stimulation of immune cells can directly lead to increased IL-2 production (i.e. a Th1-type response) [75], which may help to avoid an overt Th2 phenotype.

Further Adjuvants

As well as including an oil-in-water emulsion, compositions of the invention may include one or more further adjuvants. Such adjuvants include, but are not limited to:
A mineral-containing composition, including calcium salts and aluminum salts (or mixtures thereof). Calcium salts include calcium phosphate (e.g. the “CAP” particles disclosed in ref. 76). Aluminum salts include hydroxides, phosphates, sulfates, etc., with the salts taking any suitable form (e.g. gel, crystalline, amorphous, etc.). Adsorption to these salts is preferred. The mineral containing compositions may also be formulated as a particle of metal salt [77]. Aluminum salt adjuvants are described in more detail below.
Cytokine-inducing agents (see in more detail below).
Saponins [chapter 22 of ref. 66], which are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™. Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 78. Saponin formulations may also comprise a sterol, such as cholesterol [79]. Combinations of saponins and cholesterols can be used to form unique particles called immunostimulating complexes (ISCOMs) [chapter 23 of ref. 66]. ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 79-81. Optionally, the ISCOMS may be devoid of additional detergent [82]. A review of the development of saponin based adjuvants can be found in refs. 83 & 84.
Fatty adjuvants (see in more detail below).
Bacterial ADP-ribosylating toxins (e.g. the E. coli heat labile enterotoxin “LT”, cholera toxin “CT”, or pertussis toxin “PT”) and detoxified derivatives thereof, such as the mutant toxins known as LT-K63 and LT-R72 [85]. The use of detoxified ADP-ribosylating toxins as mucosal adjuvants is described in ref. 86 and as parenteral adjuvants in ref. 87.
Bioadhesives and mucoadhesives, such as esterified hyaluronic acid microspheres [88] or chitosan and its derivatives [89].
Microparticles (ie, a particle of ˜100 nm to ˜150 μm in diameter, more preferably ˜200 nm to ˜30 μm in diameter, and most preferably ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) being preferred, optionally treated to have a negatively-charged surface (e.g. with SDS) or a positively-charged surface (e.g. with a cationic detergent, such as CTAB).
Liposomes (Chapters 13 & 14 of ref. 66). Examples of liposome formulations suitable for use as adjuvants are described in refs. 90-92. Liposomes can elicit strong Th1 responses, particularly cationic liposomes containing mycobacterial lipids [93].
Polyoxyethylene ethers and polyoxyethylene esters [94]. Such formulations further include polyoxyethylene sorbitan ester surfactants in combination with an octoxynol [95] as well as polyoxyethylene alkyl ethers or ester surfactants in combination with at least one additional non-ionic surfactant such as an octoxynol [96]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.
Muramyl peptides, such as N-acetylmuramyl-L-threonyl-D-isoglutamine (“thr-MDP”), N-acetyl-normuramyl-L-alanyl-D-isoglutamine (nor-MDP), N-acetylglucsaminyl-N-acetylmuramyl-L-Al-D-isoglu-L-Ala-dipalmitoxy propylamide (“DTP-DPP”, or “Theramide™), N-acetylmuramyl-L-alanyl-D-isoglutaminyl-L-alanine-2-(1′-2′dipalmitoyl-sn-glycero-3-hydroxyphosphoryloxy)-ethylamine (“MTP-PE”).
Methyl inosine 5′-monophosphate (“MIMP”) [97].
Compounds containing lipids linked to a phosphate-containing acyclic backbone, such as the TLR4 antagonist E5564 [98,99]:

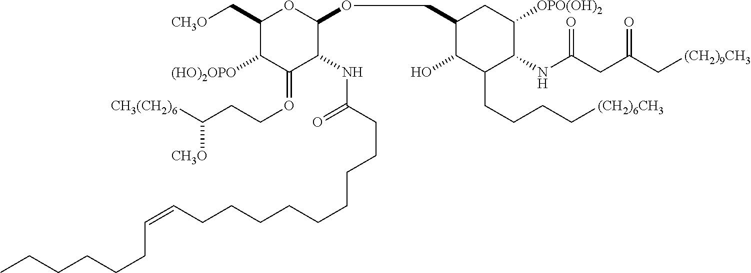

Derivatives of lipid A from Escherichia coli such as OM-174 (described in refs. 100 & 101).
A compound of formula I, II or III, or a salt thereof:

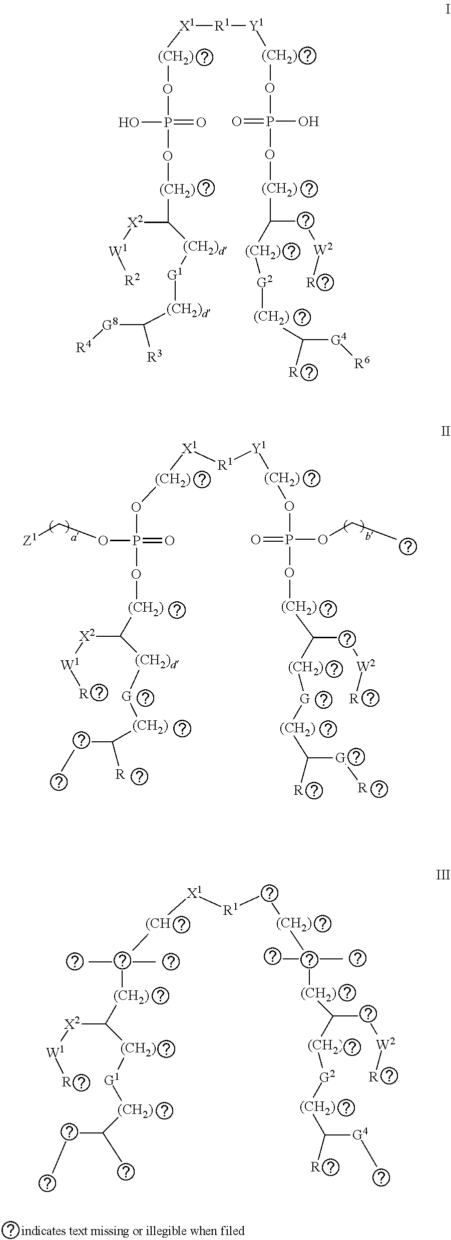

as defined in reference 102, such as ‘ER 803058’, ‘ER 803732’, ‘ER 804053’, ER 804058’, ‘ER 804059’, ‘ER 804442’, ‘ER 804680’, ‘ER 804764’, ER 803022 or ‘ER 804057’ e.g.:

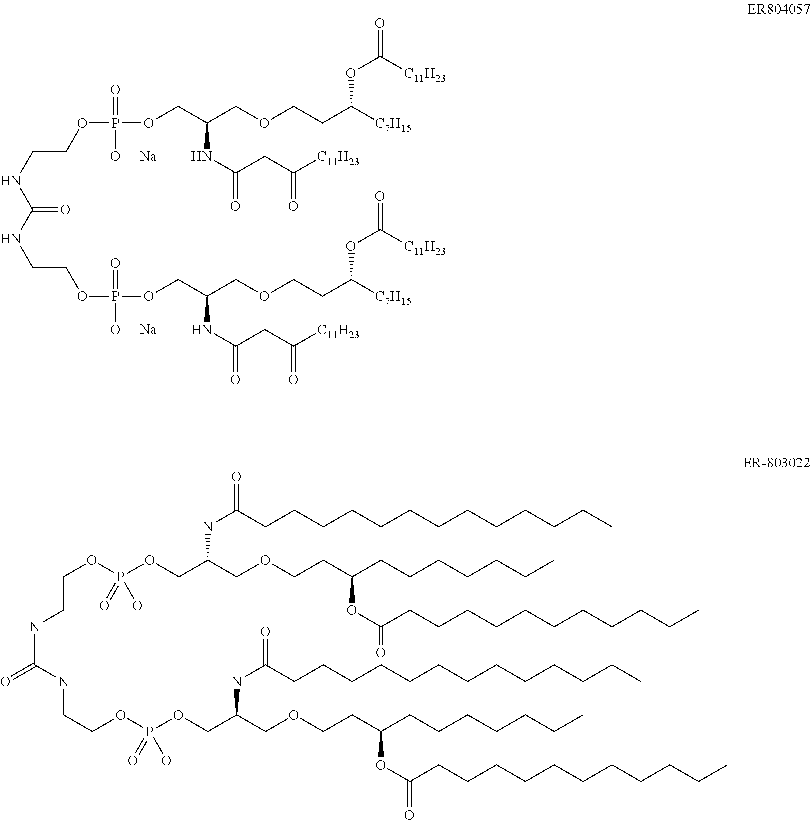

A polyhydroxlated pyrrolizidine compound [103], such as one having formula:

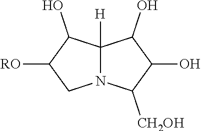

where R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof. Examples include, but are not limited to: casuarine, casuarine-6-α-D-glucopyranose, 3-epi-casuarine, 7-epi-casuarine, 3,7-diepi-casuarine, etc.
An outer membrane protein proteosome preparation prepared from a first Gram-negative bacterium in combination with a liposaccharide preparation derived from a second Gram-negative bacterium, wherein the outer membrane protein proteosome and liposaccharide preparations form a stable non-covalent adjuvant complex. Such complexes include “IVX-908”, a complex comprised of Neisseria meningitidis outer membrane and lipopolysaccharides. They have been used as adjuvants for influenza vaccines [104].
A gamma inulin [105] or derivative thereof, such as algammulin.

These and other adjuvant-active substances are discussed in more detail in references 66 & 67.

Compositions may include two or more of said adjuvants. For example, they may advantageously include both an oil-in-water emulsion and a cytokine-inducing agent, as this combination improves the cytokine responses elicited by influenza vaccines, such as the interferon-γ response, with the improvement being much greater than seen when either the emulsion or the agent is used on its own.

Antigens and adjuvants in a composition will typically be in admixture.

Preferred further adjuvants are those that favor a Th1-type immune response. Such adjuvants include, but are not limited to: immunostimulatory oligonucleotides [106]; 3dMPL [107]; ISCOMs: QS21; PLG microparticles; calcium phosphate [108]; polyhydroxlated pyrrolizidines; gamma inulins [109]; imidazoquinolines [122]; loxoribine: and aminoalkyl glucosaminide phosphate derivatives [110].

Cytokine-Inducing Agents

Cytokine-inducing agents for inclusion in compositions of the invention are able, when administered to a patient, to elicit the immune system to release cytokines, including interferons and interleukins.

Cytokine responses are known to be involved in the early and decisive stages of host defense against influenza infection [111]. Preferred agents can elicit the release of one or more of: interferon-γ; interleukin-1; interleukin-2: interleukin-12; TNF-α; TNF-β; and GM-CSF. Preferred agents elicit the release of cytokines associated with a Th1-type immune response e.g. interferon-γ, TNF-α, interleukin-2. Stimulation of both interferon-γ and interleukin-2 is preferred.

As a result of receiving a composition of the invention, therefore, a patient will have T cells that, when stimulated with an influenza antigen, will release the desired cytokine(s) in an antigen-specific manner. For example, T cells purified form their blood will release γ-interferon when exposed in vitro to influenza virus haemagglutinin. Methods for measuring such responses in peripheral blood mononuclear cells (PBMC) are known in the art, and include ELISA, ELISPOT, flow-cytometry and real-time PCR. For example, reference 112 reports a study in which antigen-specific T cell-mediated immune responses against tetanus toxoid, specifically γ-interferon responses, were monitored, and found that ELISPOT was the most sensitive method to discriminate antigen-specific TT-induced responses from spontaneous responses, but that intracytoplasmic cytokine detection by flow cytometry was the most efficient method to detect re-stimulating effects.

Suitable cytokine-inducing agents include, but are not limited to:
An immunostimulatory oligonucleotide, such as one containing a CpG motif (a dinucleotide sequence containing an unmethylated cytosine linked by a phosphate bond to a guanosine), or a double-stranded RNA, or an oligonucleotide containing a palindromic sequence, or an oligonucleotide containing a poly(dG) sequence.
3-O-deacylated monophosphoryl lipid A (‘3dMPL’, also known as ‘MPL™’) [113-116].
An imidazoquinoline compound, such as Imiquimod (“R-837”) [117,118], Resiquimod (“R-848”) [119], and their analogs; and salts thereof (e.g. the hydrochloride salts). Further details about immunostimulatory imidazoquinolines can be found in references 120 to 124.
A thiosemicarbazone compound, such as those disclosed in reference 125. Methods of formulating, manufacturing, and screening for active compounds are also described in reference 125. The thiosemicarbazones are particularly effective in the stimulation of human peripheral blood mononuclear cells for the production of cytokines, such as TNF-α.
A tryptanthrin compound, such as those disclosed in reference 126. Methods of formulating, manufacturing, and screening for active compounds are also described in reference 126. The thiosemicarbazones are particularly effective in the stimulation of human peripheral blood mononuclear cells for the production of cytokines, such as TNF-α.
A nucleoside analog, such as: (a) Isatorabine (ANA-245: 7-thia-8-oxoguanosine):

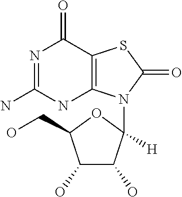 [SMILES: Nc1nc(=O)c2sc(=O)n([C@@H]3O[C@H](CO)[C@@H](O)[C@H]3O)c2[nH]1]

and prodrugs thereof; (b) ANA975; (c) ANA-025-1; (d) ANA380; (e) the compounds disclosed in references 127 to 129; (f) a compound having the formula:

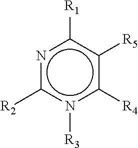

wherein:
R₁ and R₂ are each independently H, halo, —NRₐR_{b}, —OH, C₁₋₆ alkoxy, substituted C₁₋₆ alkoxy, heterocvclyl, substituted heterocyclyl, C₆₋₁₀ aryl, substituted C₆₋₁₀ aryl, C₁₋₆ alkyl, or substituted C₁₋₆ alkyl;
R₃ is absent, H, C₁₋₆ alkyl, substituted C₁₋₆ alkyl, C₆₋₁₀ aryl, substituted C₆₋₁₀ aryl, heterocyclyl, or substituted heterocyclyl;
R₄ and R₅ are each independently H, halo, heterocyclyl, substituted heterocyclyl. —C(O)—R_{d}, C₁₋₄ alkyl, substituted C₁₋₆ alkyl, or bound together to form a 5 membered ring as in R₄₋₅:

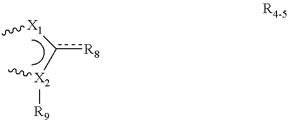

the binding being achieved at the bonds indicated by a [symbol]
X₁ and X₂ are each independently N, C, O, or S;
R₈ is H, halo, —OH, C₁₋₆ alkyl, C₂₋₆ alkenyl, C₂₋₆ alkynyl, —OH, —NRₐR_{b}, —(CH₂)—O—R_{c}, —O—(C₁₋₆ alkyl), —S(O)ₚRₑ, or —C(O)—R_{d};
R₉ is H, C₁₋₆ alkyl, substituted C₁₋₆ alkyl, heterocyclyl, substituted heterocyclyl or R₉, wherein R₉, is:

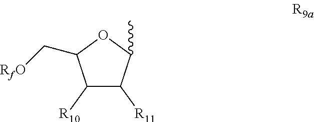

the binding being achieved at the bond indicated by a [symbol]
R₁₀ and R₁₁ are each independently H, halo, C₁₋₆ alkoxy, substituted C₁₋₆ alkoxy, —NRₐR_{b}, or —OH;
each Rₐ and R_{b} is independently H, C₁₋₆ alkyl, substituted C₁₋₆ alkyl, —C(O)R_{d}, C₆₋₁₀ aryl;
each R_{c} is independently H, phosphate, diphosphate, triphosphate, C₁₋₆ alkyl, or substituted C₁₋₆ alkyl;
each R_{d} is independently H, halo, C₁₋₆ alkyl, substituted C₁₋₆ alkyl, C₁₋₆ alkoxy, substituted C₁₋₆ alkoxy, —NH₂, —NH(C₁₋₆ alkyl), —NH(substituted C₁₋₆ alkyl), —N(C₁₋₆ alkyl)₂, —N(substituted C₁₋₆ alkyl)₂, C₆₋₁₀ aryl, or heterocyclyl;
each Rₑ is independently H, C₁₋₄ alkyl, substituted C₁₋₄ alkyl, C₆₋₁₀ aryl, substituted C₆₋₁₀ aryl, heterocyclyl, or substituted heterocyclyl;
each R_{f} is independently H, C₁₋₆ alkyl, substituted C₁₋₆ alkyl, —C(O)R_{d}, phosphate, diphosphate, or triphosphate;
each n is independently 0, 1, 2, or 3;
each p is independently 0, 1, or 2; or
or (g) a pharmaceutically acceptable salt of any of (a) to (f), a tautomer of any of (a) to (f), or a pharmaceutically acceptable salt of the tautomer.
Loxoribine (7-allyl-8-oxoguanosine) [130].
Compounds disclosed in reference 131, including: Acylpiperazine compounds, Indoledione compounds, Tetrahydraisoquinoline (THIQ) compounds, Benzocyclodione compounds, Aminoazavinyl compounds, Aminobenzimidazole quinolinone (ABIQ) compounds [132,133]. Hydrapthalamide compounds, Benzophenone compounds, Isoxazole compounds. Sterol compounds, Quinazilinone compounds, Pyrrole compounds [134], Anthraquinone compounds, Quinoxaline compounds, Triazine compounds, Pyrazalopyrimidine compounds, and Benzazole compounds [135].
A polyoxidonium polymer [136,137] or other N-oxidized polyethylene-piperazine derivative.
Compounds disclosed in reference 138.
An aminoalkyl glucosaminide phosphate derivative, such as RC-529 [139,140].
A phosphazene, such as poly[di(carboxylatophenoxy)phosphazene] (“PCPP”) as described, for example, in references 141 and 142.
Small molecule immunopotentiators (SMIPs) such as:
N2-methyl-1-(2-methylpropyl)-1H-imidazo[4,5-c]quinoline-2,4-diamine;
N2,N2-dimethyl-1-(2-methylpropyl)-1H-imidazo[4,5-c]quinoline-2,4-diamine:
N2-ethyl-N2-methyl-1-(2-methylpropyl)-1H-imidazo[4,5-c]quinoline-2,4-diamine;
N2-methyl-1-(2-methylpropyl)-N2-propyl-1H-imidazo[4,5-c]quinoline-2,4-diamine;
1-(2-methylpropyl)-N2-propyl-1H-imidazo[4,5-c]quinoline-2,4-diamine;
N2-butyl-1-(2-methylpropyl)-1H-imidazo[4,5-c] quinoline-2,4-diamine;
N2-butyl-N2-methyl-1-(2-methylpropyl)-1H-imidazo[4,5-c]quinoline-2,4-diamine;
N2-methyl-1-(2-methylpropyl)-N2-pentyl-1H-imidazo[4,5-c]quinoline-2,4-diamine:
N2-methyl-1-(2-methylpropyl)-N2-prop-2-enyl-1H-imidazo[4,5-c]quinoline-2,4-diamine;
1-(2-methylpropyl)-2-[(phenylmethyl)thio]-1H-imidazo[4,5-c]quinolin-4-amine;
1-(2-methylpropyl)-2-(propylthio)-1H-imidazo[4,5-c]quinolin-4-amine;
2-[14-amino-1-(2-methylpropyl)-1H-imidazo[4,5-c]quinolin-2-yl(methyl)amino]ethanol:
2-[[4-amino-1-(2-methylpropyl)-1H-imidazo[4,5-c]quinolin-2-yl](methyl)amino]ethyl acetate:
4-amino-1-(2-methylpropyl)-1,3-dihydro-2H-imidazo[4,5-c]quinolin-2-one;
N2-butyl-1-(2-methylpropyl)-N4,N4-bis(phenylmethyl)-1H-imidazo[4,5-c]quinoline-2,4-diamine;
N2-butyl-N2-methyl-1-(2-methylpropyl)-N4,N4-bis(phenylmethyl)-1H-imidazo[4,5-c]quinoline-2,4-diamine;
N2-methyl-1-(2-methylpropyl)-N4,N4-bis(phenylmethyl)-1H-imidazo[4,5-c]quinoline-2,4-diamine;
N2,N2-dimethyl-1-(2-methylpropyl)-N4,N4-bis(phenylmethyl)-1H-imidazo[4,5-c]quinoline-2,4-diamine:
1-(4-amino-2-[methyl(propyl)amino]-1H-imidazo[4,5-c]quinolin-1-yl-2-methylpropan-2-ol;
1-[4-amino-2-(propylamino)-1H-imidazo[4,5-c]quinolin-1-yl]-2-methylpropan-2-ol;
N4,N4-dibenzyl-1-(2-methoxy-2-methylpropyl)-N2-propyl-1H-imidazo[4,5-c]quinoline-2,4-diamine.

The cytokine-inducing agents for use in the present invention may be modulators and/or agonists of Toll-Like Receptors (TLR). For example, they may be agonists of one or more of the human TLR1, TLR2, TLR3, TLR4, TLR7, TLR8, and/or TLR9 proteins. Preferred agents are agonists of TLR7 (e.g. imidazoquinolines) and/or TLR9 (e.g. CpG oligonucleotides). These agents are useful for activating innate immunity pathways.

The cytokine-inducing agent can be added to a composition at various stages during its production. For example, it may be within an antigen composition, and this mixture can then be added to an oil-in-water emulsion. As an alternative, it may be within an oil-in-water emulsion, in which case the agent can either be added to the emulsion components before emulsification, or it can be added to the emulsion after emulsification. Similarly, the agent may be coacervated within the emulsion droplets. The location and distribution of the cytokine-inducing agent within the final composition will depend on its hydrophilic/lipophilic properties e.g. the agent can be located in the aqueous phase, in the oil phase, and/or at the oil-water interface.

The cytokine-inducing agent can be conjugated to a separate agent, such as an antigen (e.g. CRM 197). A general review of conjugation techniques for small molecules is provided in ref. 143. As an alternative, the adjuvants may be non-covalently associated with additional agents, such as by way of hydrophobic or ionic interactions.

Two preferred cytokine-inducing agents are (a) immunostimulatory oligonucleotides and (b) 3dMPL.

Immunostimulatory Oligonucleotides

Immunostimulatory oligonucleotides can include nucleotide modifications/analogs such as phosphorothioate modifications and can be double-stranded or (except for RNA) single-stranded. References 144, 145 and 146 disclose possible analog substitutions e.g. replacement of guanosine with 2′-deoxy-7-deazaguanosine. The adjuvant effect of CpG oligonucleotides is further discussed in refs. 147-152. A CpG sequence may be directed to TLR9, such as the motif GTCGTT or TTCGTT [153]. The CpG sequence may be specific for inducing a Th1 immune response, such as a CpG-A ODN (oligodeoxynucleotide), or it may be more specific for inducing a B cell response, such a CpG-B ODN. CpG-A and CpG-B ODNs are discussed in refs. 154-156. Preferably, the CpG is a CpG-A ODN. Preferably, the CpG oligonucleotide is constructed so that the 5′ end is accessible for receptor recognition. Optionally, two CpG oligonucleotide sequences may be attached at their 3′ ends to form “immunomers”. See, for example, references 153 & 157-159. A useful CpG adjuvant is CpG7909, also known as ProMune™ (Coley Pharmaceutical Group, Inc.).

As an alternative, or in addition, to using CpG sequences, TpG sequences can be used [160]. These oligonucleotides may be free from unmethylated CpG motifs.

The immunostimulatory oligonucleotide may be pyrimidine-rich. For example, it may comprise more than one consecutive thymidine nucleotide (e.g. TTTT, as disclosed in ref. 160), and/or it may have a nucleotide composition with >25% thymidine (e.g. >35%, >40%, >50%, >60%, >80%, etc.). For example, it may comprise more than one consecutive cytosine nucleotide (e.g. CCCC, as disclosed in ref. 160), and/or it may have a nucleotide composition with >25% cytosine (e.g. >35%, >40%, >50%, >60%, >80%, etc.). These oligonucleotides may be free from unmethylated CpG motifs.

Immunostimulatory oligonucleotides will typically comprise at least 20 nucleotides. They may comprise fewer than 100 nucleotides.

A combination of liposomes and immunostimulatory oligonucleotides can be used, particularly where the oligonucleotides are encapsulated within the liposomes. This combination can induce strong Th1 immune responses [161].

3dMPL

3dMPL (also known as 3 de-O-acylated monophosphoryl lipid A or 3-O-desacyl-4′-monophosphoryl lipid A) is an adjuvant in which position 3 of the reducing end glucosamine in monophosphoryl lipid A has been de-acylated. 3dMPL has been prepared from a heptoseless mutant of Salmonella minnesota, and is chemically similar to lipid A but lacks an acid-labile phosphoryl group and a base-labile acyl group. It activates cells of the monocyte/macrophage lineage and stimulates release of several cytokines, including IL-1, IL-12, TNF-α and GM-CSF (see also ref. 162). Preparation of 3dMPL was originally described in reference 163.

3dMPL can take the form of a mixture of related molecules, varying by their acylation (e.g. having 3, 4, 5 or 6 acyl chains, which may be of different lengths). The two glucosamine (also known as 2-deoxy-2-amino-glucose) monosaccharides are N-acylated at their 2-position carbons (i.e. at positions 2 and 2′), and there is also O-acylation at the 3′ position. The group attached to carbon 2 has formula —NH—CO—CH₂—CR¹R^{1′}. The group attached to carbon 2′ has formula —NH—CO—CH₂—CR²R^{2′}. The group attached to carbon 3′ has formula —O—CO—CH₂—CR³R^{3′}. A representative structure is:

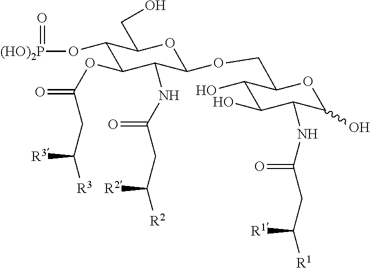

Groups R¹, R² and R³ are each independently —(CH₂)ₙ—CH₃. The value of n is preferably between 8 and 16, more preferably between 9 and 12, and is most preferably 10.

Groups R_{1′}, R^{2′} and R^{3′} can each independently be: (a) —H; (b) —OH; or (c) —O—CO—R₄, where R⁴ is either —H or —(CH₂)ₘ—CH₃, wherein the value of m is preferably between 8 and 16, and is more preferably 10, 12 or 14. At the 2 position, m is preferably 14. At the 2′ position, m is preferably 10. At the 3′ position, m is preferably 12. Groups R^{1′}, R^{2′} and R^{3′} are thus preferably —O-acyl groups from dodecanoic acid, tetradecanoic acid or hexadecanoic acid.

When all of R^{1′}, R^{2′} and R^{3′} are —H then the 3dMPL has only 3 acyl chains (one on each of positions 2, 2′ and 3′). When only two of R^{1′}, R^{2′} and R^{3′} are —H then the 3dMPL can have 4 acyl chains. When only one of R^{1′}, R^{2′} and R^{3′} is —H then the 3dMPL can have 5 acyl chains. When none of R^{1′}, R^{2′} and R^{3′} is —H then the 3dMPL can have 6 acyl chains. The 3dMPL adjuvant used according to the invention can be a mixture of these forms, with from 3 to 6 acyl chains, but it is preferred to include 3dMPL with 6 acyl chains in the mixture, and in particular to ensure that the hexaacyl chain form makes up at least 10% by weight of the total 3dMPL e.g. ≥20%, ≥30%, ≥407%, ≥50% or more. 3dMPL with 6 acyl chains has been found to be the most adjuvant-active form.

Thus the most preferred form of 3dMPL for inclusion in compositions of the invention is:

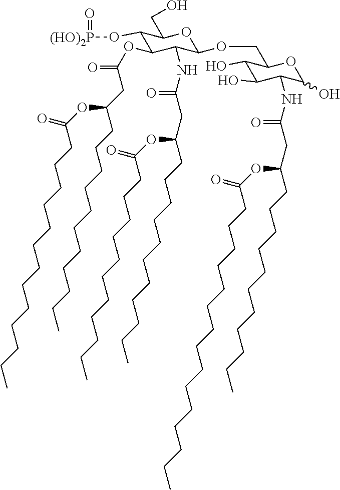

Where 3dMPL is used in the form of a mixture then references to amounts or concentrations of 3dMPL in compositions of the invention refer to the combined 3dMPL species in the mixture.

In aqueous conditions, 3dMPL can form micellar aggregates or particles with different sizes e.g. with a diameter <150 nm or >500 nm. Either or both of these can be used with the invention, and the better particles can be selected by routine assay. Smaller particles (e.g. small enough to give a clear aqueous suspension of 3dMPL) are preferred for use according to the invention because of their superior activity [164]. Preferred particles have a mean diameter less than 220 nm, more preferably less than 200 nm or less than 150 nm or less than 120 nm, and can even have a mean diameter less than 100 nm. In most cases, however, the mean diameter will not be lower than 50 nm. These particles are small enough to be suitable for filter sterilization. Particle diameter can be assessed by the routine technique of dynamic light scattering, which reveals a mean particle diameter. Where a particle is said to have a diameter of x nm, there will generally be a distribution of particles about this mean, but at least 50% by number (e.g. ≥60%, ≥70%, ≥80%, ≥90%, or more) of the particles will have a diameter within the range x±25%.

3dMPL can advantageously be used in combination with an oil-in-water emulsion. Substantially all of the 3dMPL may be located in the aqueous phase of the emulsion.

A typical amount of 3dMPL in a vaccine is 10-100 μg/dose e.g. about 25 μg or about 50 μg.

The 3dMPL can be used on its own, or in combination with one or more further compounds. For example, it is known to use 3dMPL in combination with the QS21 saponin [165] (including in an oil-in-water emulsion [166]), with an immunostimulatory oligonucleotide, with both QS21 and an immunostimulatory oligonucleotide, with aluminum phosphate [167], with aluminum hydroxide [168], or with both aluminum phosphate and aluminum hydroxide.

Pharmaceutical Compositions

Compositions of the invention are pharmaceutically acceptable. They may include components in addition to the split antigen and emulsion e.g. they typically include one or more pharmaceutical carrier(s) and/or excipient(s). A thorough discussion of such components is available in ref. 169.

Compositions will generally be in aqueous form. The split antigen and emulsion will typically be in admixture.

The composition may include preservatives such as thiomersal or 2-phenoxyethanol. It is preferred, however, that the vaccine should be substantially free from (i.e. less than 5 μg/ml) mercurial material e.g. thiomersal-free [14,170]. Vaccines containing no mercury are more preferred, and this can conveniently be achieved when using a tocopherol-containing adjuvant by following ref. 14. Preservative-free vaccines are particularly preferred.

To control tonicity, it is preferred to include a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, and will more preferably fall within the range of 290-310 mOsm/kg. Osmolality has previously been reported not to have an impact on pain caused by vaccination [171], but keeping osmolality in this range is nevertheless preferred.

Compositions may include one or more buffers. Typical buffers include: a phosphate buffer: a Tris buffer; a borate buffer; a succinate buffer; a histidine buffer: or a citrate buffer. Buffers will typically be included in the 5-20 mM range. An emulsion formed in phosphate-buffered saline can conveniently be used.

The pH of a composition will generally be between 5.0 and 8.1, and more typically between 6.0 and 8.0 e.g. 6.5 and 7.5, or between 7.0 and 7.8. A process of the invention may therefore include a step of adjusting the pH of the bulk vaccine prior to packaging.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunisation, or may include material for multiple immunisations (i.e. a ‘multidose’ kit). The inclusion of a preservative is preferred in multidose arrangements. As an alternative (or in addition) to including a preservative in multidose compositions, the compositions may be contained in a container having an aseptic adaptor for removal of material.

Influenza vaccines are typically administered in a dosage volume of about 0.5 ml, although a half dose (i.e. about 0.25 ml) may be administered e.g. to children.

Compositions and kits are preferably stored at between 2° C. and 8° C. They should not be frozen.

They should ideally be kept out of direct light.

Kits of the Invention

Compositions of the invention may be prepared extemporaneously, at the time of delivery. Thus the invention provides kits including the various components ready for mixing. The kit allows the adjuvant and the antigen to be kept separately until the time of use, which can be useful when using an oil-in-water emulsion adjuvant.

The components are physically separate from each other within a kit, and this separation can be achieved in various ways. For instance, the two components may be in two separate containers, such as vials. The contents of the two vials can then be mixed e.g. by removing the contents of one vial and adding them to the other vial, or by separately removing the contents of both vials and mixing them in a third container.

In a preferred arrangement, one of the kit components is in a syringe and the other is in a container such as a vial. The syringe can be used (e.g. with a needle) to insert its contents into the second container for mixing, and the mixture can then be withdrawn into the syringe. The mixed contents of the syringe can then be administered to a patient, typically through a new sterile needle. Packing one component in a syringe eliminates the need for using a separate syringe for patient administration.

In another preferred arrangement, the two kit components are held together but separately in the same syringe e.g. a dual-chamber syringe, such as those disclosed in references 172-179 etc. When the syringe is actuated (e.g. during administration to a patient) then the contents of the two chambers are mixed. This arrangement avoids the need for a separate mixing step at the time of use.

The kit components will generally be in aqueous form. In some arrangements, a component (typically the antigen component rather than the adjuvant component) is in dry form (e.g. in a lyophilised form), with the other component being in aqueous form. The two components can be mixed in order to reactivate the dry component and give an aqueous composition for administration to a patient. A lyophilised component will typically be located within a vial rather than a syringe.

Dried components may include stabilizers such as lactose, sucrose or mannitol, as well as mixtures thereof e.g. lactose/sucrose mixtures, sucrose/mannitol mixtures, etc. One possible arrangement uses an aqueous adjuvant component in a pre-filled syringe and a lyophilised antigen component in a vial.

Packaging of Compositions or Kit Components

Suitable containers for compositions of the invention (or kit components) include vials, syringes (e.g. disposable syringes), nasal sprays, etc. These containers should be sterile.

Where a composition/component is located in a vial, the vial may be made of a glass or plastic material. It can be sterilized before the composition/component is added to it. To avoid problems with latex-sensitive patients, vials may be sealed with a latex-free stopper, and the absence of latex in all packaging material is preferred. The vial may include a single dose of vaccine, or it may include more than one dose (a ‘multidose’ vial) e.g. 10 doses. Preferred vials are made of colorless glass.

A vial can have a cap (e.g. a Luer lock) adapted such that a pre-filled syringe can be inserted into the cap, the contents of the syringe can be expelled into the vial (e.g. to reconstitute lyophilised material therein), and the contents of the vial can be removed back into the syringe. After removal of the syringe from the vial, a needle can then be attached and the composition can be administered to a patient. The cap is preferably located inside a seal or cover, such that the seal or cover has to be removed before the cap can be accessed. A vial may have a cap that permits aseptic removal of its contents, particularly for multidose vials.

Where a composition/component is packaged into a syringe, the syringe will not normally have a needle attached to it, although a separate needle may be supplied with the syringe for assembly and use. Safety needles are preferred. 1-inch 23-gauge, 1-inch 25-gauge and ⅝-inch 25-gauge needles are typical. Syringes may be provided with peel-off labels on which the lot number, influenza season and expiration date of the contents may be printed, to facilitate record keeping. The plunger in the syringe preferably has a stopper to prevent the plunger from being accidentally removed during aspiration. The syringes may have a latex rubber cap and/or plunger. Disposable syringes contain a single dose of vaccine. The syringe will generally have a tip cap to seal the tip prior to attachment of a needle, and the tip cap is preferably made of a butyl rubber. If the syringe and needle are packaged separately then the needle is preferably fitted with a butyl rubber shield. Preferred syringes are those marketed under the trade name “Tip-Lok” ™.

Containers may be marked to show a half-dose volume e.g. to facilitate delivery to children. For instance, a syringe containing a 0.5 ml dose may have a mark showing a 0.25 ml volume.

Where a glass container (e.g. a syringe or a vial) is used, then it is preferred to use a container made from a borosilicate glass rather than from a soda lime glass.

A kit or composition may be packaged (e.g. in the same box) with a leaflet including details of the vaccine e.g. instructions for administration, details of the antigens within the vaccine, etc. The instructions may also contain warnings e.g. to keep a solution of adrenaline readily available in case of anaphylactic reaction following vaccination, etc.

### PREFERRED EMBODIMENT OF THE INVENTION

A preferred composition comprises (i) an oil-in-water emulsion including squalene and polysorbate 80, and (ii) a split influenza virus antigen.

A preferred kit comprises (i) a first kit component comprising a split influenza virus antigen, and (ii) a second kit component comprising an oil-in-water emulsion that includes squalene and polysorbate 80.

A preferred process comprises the steps of combining: (i) a split influenza virus antigen; and (ii) an oil-in-water emulsion, wherein the emulsion includes squalene and polysorbate 80.

Before the process is performed, the concentrations of antigen and emulsion are higher than desired for the final product, because the combination of the separate components causes dilution. If substantially equal volumes of the two components are mixed, for instance, then the pre-mixing concentrations will be double the desired final concentrations.

The split influenza virus antigen and the emulsion will thus be prepared separately and then combined. Although preparation of the two components may be performed at different times by different people in different places, the invention provides a process comprising the steps of: (i) preparing a split influenza virus antigen; (ii) preparing an oil-in-water emulsion, wherein the emulsion includes squalene and polysorbate 80; and (iii) combining the split influenza virus antigen and the oil-in-water emulsion. The emulsion can be prepared by combining oil(s) and surfactant(s) in an aqueous medium and then microfluidizing the combination to form the emulsion e.g. to give sub-micron droplets.

Where antigen and emulsion are combined on an industrial scale then the process can include a further step of extracting a unit dose of the mixture.

The split influenza virus antigen may be monovalent or multivalent (such as a trivalent e.g. from two influenza A viruses and one influenza B virus).

In addition to squalene and polysorbate 80, the emulsion may include one or more of: (a) Span 85: (b) a tocopherol; (c) a polyoxyethanol, such as Triton X-100 (octylphenoxypolyethoxyethanol): (d) a citrate buffer: and/or (e) a phosphate buffer.

Methods of Treatment and Administration of the Vaccine

Compositions of the invention are suitable for administration to human patients, and the invention provides a method of raising an immune response in a patient, comprising the step of administering a composition of the invention to the patient.

The invention also provides a kit or composition of the invention for use as a medicament.

The invention also provides the use of (i) a split influenza virus antigen and (ii) an oil-in-water emulsion that includes free surfactant in its aqueous phase, in the manufacture of a medicament for raising an immune response in a patient.

The immune response raised by these methods and uses will generally include an antibody response, preferably a protective antibody response. Methods for assessing antibody responses, neutralising capability and protection after influenza virus vaccination are well known in the art. Human studies have shown that antibody titers against hemagglutinin of human influenza virus are correlated with protection (a serum sample hemagglutination-inhibition titer of about 30-40 gives around 50% protection from infection by a homologous virus) [180]. Antibody responses are typically measured by hemagglutination inhibition, by microneutralisation, by single radial immunodiffusion (SRID), and/or by single radial hemolysis (SRH). These assay techniques are well known in the art.

Compositions of the invention can be administered in various ways. The most preferred immunisation route is by intramuscular injection (e.g. into the arm or leg), but other available routes include subcutaneous injection, intranasal [181-183], oral [184], intradermal [185,186], transcutaneous, transdermal [187], etc.

Vaccines prepared according to the invention may be used to treat both children and adults. Influenza vaccines are currently recommended for use in pediatric and adult immunisation, from the age of 6 months. Thus the patient may be less than 1 year old, 1-5 years old, 5-15 years old, 15-55 years old, or at least 55 years old. Preferred patients for receiving the vaccines are the elderly (e.g. ≥50 years old. ≥60 years old, preferably ≥65 years), the young (e.g. ≤5 years old), hospitalised patients, healthcare workers, armed service and military personnel, pregnant women, the chronically ill, immunodeficient patients, patients who have taken an antiviral compound (e.g. an oseltamivir or zanamivir compound: see below) in the 7 days prior to receiving the vaccine, people with egg allergies and people travelling abroad. The vaccines are not suitable solely for these groups, however, and may be used more generally in a population. For pandemic strains, administration to all age groups is preferred.

Preferred compositions of the invention satisfy 1, 2 or 3 of the CPMP criteria for efficacy. In adults (18-60 years), these criteria are: (1) ≥70% seroprotection; (2) ≥40% seroconversion; and/or (3) a GMT increase of ≥2.5-fold. In elderly (>60 years), these criteria are: (1) ≥60% seroprotection. (2) ≥30% seroconversion; and/or (3) a GMT increase of ≥2-fold. These criteria are based on open label studies with at least 50 patients.

Treatment can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Administration of more than one dose (typically two doses) is particularly useful in immunologically naïve patients e.g. for people who have never received an influenza vaccine before, or for vaccinating against a new HA subtype (as in a pandemic outbreak). Multiple doses will typically be administered at least 1 week apart (e.g. about 2 weeks, about 3 weeks, about 4 weeks, about 6 weeks, about 8 weeks, about 10 weeks, about 12 weeks, about 16 weeks, etc.).

Vaccines produced by the invention may be administered to patients at substantially the same time as (e.g. during the same medical consultation or visit to a healthcare professional or vaccination centre) other vaccines e.g. at substantially the same time as a measles vaccine, a mumps vaccine, a rubella vaccine, a MMR vaccine, a varicella vaccine, a MMRV vaccine, a diphtheria vaccine, a tetanus vaccine, a pertussis vaccine, a DTP vaccine, a conjugated H. influenzae type b vaccine, an inactivated poliovirus vaccine, a hepatitis B virus vaccine, a meningococcal conjugate vaccine (such as a tetravalent A-C-W135-Y vaccine), a respiratory syncytial virus vaccine, a pneumococcal conjugate vaccine, etc. Administration at substantially the same time as a pneumococcal vaccine or a meningococcal vaccine is particularly useful in elderly patients.

Similarly, vaccines of the invention may be administered to patients at substantially the same time as (e.g. during the same medical consultation or visit to a healthcare professional) an antiviral compound, and in particular an antiviral compound active against influenza virus (e.g. oseltamivir and/or zanamivir). These antivirals include neuraminidase inhibitors, such as a (3R,4R,5S)-4-acetylamino-5-amino-3(1-ethylpropoxy)-1-cyclohexene-1-carboxylic acid or 5-(acetylamino)-4-[(aminoiminomethyl)-amino]-2,6-anhydro-3,4,5-trideoxy-D-glycero-D-galactonon-2-enonic acid, including esters thereof (e.g. the ethyl esters) and salts thereof (e.g. the phosphate salts). A preferred antiviral is (3R,4R,5S)-4-acetylamino-5-amino-3(1-ethylpropoxy)-1-cyclohexene-1-carboxylic acid, ethyl ester, phosphate (1:1), also known as oseltamivir phosphate (TAMIFLU™).

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encaphalopathies (TSEs), and in particular free from bovine spongiform encephalopathy (BSE). Overall, it is preferred to culture cells in the total absence of animal-derived materials.

Where a compound is administered to the body as part of a composition then that compound may alternatively be replaced by a suitable prodrug.

Where a cell substrate is used for reassortment or reverse genetics procedures, it is preferably one that has been approved for use in human vaccine production e.g. as in Ph Eur general chapter 5.2.3.

### MODES FOR CARRYING OUT THE INVENTION

Analysis of Free Surfactant in a Squalene-in-Water Emulsion

A microfluidised squalene-in-water emulsion adjuvant comprising a Tween 80 surfactant was prepared as disclosed in chapter 10 of ref. 66. The emulsion was analysed to determine the level of Tween 80 in its aqueous phase. The oil phase of the adjuvant was removed, and the esters in the aqueous phase were saponified and fluorescently derivatised. After chromatographic separation, fluorescence detection was used to quantify the total amount of Tween 80 in the aqueous phase.

A RP-HPLC method was also used to quantify Tween 80 in the separated aqueous phase.

Both methods gave similar results, with 12±1% of the total Tween 80 in the emulsion being found in the aqueous phase.

Adjuvanting of Split Vaccines with MF59

Two commercially available unadjuvanted split virion trivalent influenza vaccines (“SPLIT (A)” and “SPLIT (B)” were obtained and used to immunize mice. The vaccines were diluted to give a dose of 0.2 μg each HA. Dilution used either buffer alone, or buffer and the squalene-in-water emulsion. Groups of 8 female Balb/C mice, 8 weeks old, were immunized intramuscularly with the unadjuvanted and adjuvanted vaccines, with 50 μl doses on days 0 and 28. Sera were obtained on days 14 and 42, and were analysed for anti-HA titer (IgG), HI titer and T cells. Serum IgG antibody titers (ELISA) were as follows, looking at each virus separately:

| | |
|---|---|
| Day 14 | Day 42 |

| | | | |
|---|---|---|---|
| Plain | O/W emulsion | Plain | O/W emulsion |

Anti-H1N1

| | | | | |
|---|---|---|---|---|
| SPLIT (A) | 152 | 450 | 749 | 7690 |
| SPLIT (B) | 85 | 629 | 1175 | 7738 |

Anti-H3N2

| | | | | |
|---|---|---|---|---|
| SPLIT (A) | 123 | 318 | 412 | 4583 |
| SPLIT (B) | 95 | 552 | 1111 | 6005 |

Anti-B

| | | | | |
|---|---|---|---|---|
| SPLIT (A) | 238 | 710 | 707 | 8716 |
| SPLIT (B) | 200 | 1063 | 1585 | 13682 |

HI serum antibody titers at day 42 were as follows:

| | |
|---|---|
| Plain | O/W emulsion |

Anti-H1N1

| | | |
|---|---|---|
| SPLIT (A) | 140 | 800 |
| SPLIT (B) | 285 | 1300 |

Anti-H3N2

| | | |
|---|---|---|
| SPLIT (A) | 290 | 510 |
| SPLIT (B) | 380 | 460 |

Anti-B

| | | |
|---|---|---|
| SPLIT (A) | 280 | 1560 |
| SPLIT (B) | 550 | 2280 |

Thus oil-in-water emulsions can enhance the immune responses achieved by split influenza vaccines. By including free surfactant in the aqueous phase, the emulsion can also continue to exert a ‘splitting effect’ on the virus, thereby disrupting any unsplit virions and/or virion aggregates that might otherwise be present.

It will be understood that the invention has been described by way of example only and modifications may be made whilst remaining within the scope and spirit of the invention.

### REFERENCES (THE CONTENTS OF WHICH ARE HEREBY INCORPORATED BY REFERENCE)

[1] Vaccines. (eds. Plotkin & Orenstein). 4th edition, 2004, ISBN: 0-7216-9688-0.
[2] Scheifele et al. (2003) Clin Infect Dis 36:850-7.
[3] Babiuk et al. (2004)J Med Virol 72:138-42.
[4] Skowronski et al. (2003) Jlnfect Dis 187:495-9.
[5] U.S. Pat. No. 6,372,223.
[6] WO00/15251.
[7] WO01/22992.
[8] Hehme et al. (2004) Virus Res. 103(1-2): 163-71.
[9] Huckriede et al. (2003) Methods Enzymol 373:74-91.
[10] WO02/28422.
[11] WO02/067983.
[12] WO02/074336.
WO01/21151.
[14] WO02/097072.
[15] WO2005/113756.
[16] World Health Organisation (2005) Emerging Infectious Diseases 11(10):1515-21.
[17] Herlocher et al. (2004) J Infect Dis 190(9): 1627-30.
[18] Le et al. (2005) Nature 437(7062):1108.
[19] Hoffmann et al. (2002) Vaccine 20:3165-3170.
[20] Subbarao et al. (2003) Virology 305:192-200.
[21] Liu et al. (2003) Virology 314:580-590.
[22] Ozaki et al. (2004) J. Virol. 78:1851-1857.
[23] Webby et al. (2004) Lancet 363:1099-1103.
[24] WO00/60050.
[25] WO01/04333.
[26] U.S. Pat. No. 6,649,372.
[27] Neumann et al. (2005) Proc Natl Acad Sci USA 102:16825-9.
[28] WO2006/067211.
[29] WO01/83794.
[30] Hoffmann et al. (2000) Virology 267(2):310-7.
[31] WO97/37000.
[32] Brands et al. (1999) Dev Biol Stand 98:93-100.
[33] Halperin et al. (2002) Vaccine 20:1240-7.
[34] Tree et al. (2001) Vaccine 19:3444-50.
[35] Kistner et al. (1998) Vaccine 16:960-8.
[36] Kistner et al. (1999) Dev Biol Stand 98:101-110.
[37] Bruhl et al. (2000) Vaccine 19:1149-58.
[38] Pau et al. (2001) Vaccine 19:2716-21.
[39] http://www.atcc.org/
[40] http://locus.umdnj.edu/
[41] WO03/076601.
[42] WO2005/042728.
[43] WO03/043415.
[44] WO01/85938.
[45] WO2006/108846.
[46] EP-A-1260581 (WO01/64846).
[47] WO2006/071563.
[48] WO2005/113758.
[49] WO03/023021
[50] WO03/023025
[51] WO97/37001.
[52] WO2006/027698.
[53] Lundblad (2001) Biotechnology and Applied Biochemistry 34: 195-197.
[54] Guidance for Industry: Bioanalytical Method Validation. U.S. Department of Health and Human Services Food and Drug Administration Center for Drug Evaluation and Research (CDER) Center for Veterinary Medicine (CVM). May 2001.
[55] Ji et al. (2002) Biotechniques. 32:1162-7.
[56] Briggs (1991) J Parenter Sci Technol. 45:7-12.
[157] Lahijani et al. (1998) Hum Gene Ther. 9:1173-80.
[58] Lokteff et al. (2001) Biologicals. 29:123-32.
[59] EP-B-0870508.
[60] U.S. Pat. No. 5,948,410.
[61] International patent application entitled “CELL-DERIVED VIRAL VACCINES WITH LOW LEVELS OF RESIDUAL CELL DNA”, filed 1st November 2006 claiming priority US-60/732,786.
[62] Treanor et al. (1996) J Infect Dis 173:1467-70.
[63] Keitel et al. (1996) Clin Diagn Lab Immunol 3:507-10.
[64] WO90/14837.
[65] Podda & Del Giudice (2003) Expert Rev Vaccines 2:197-203.
[66] Podda (2001) Vaccine 19: 2673-2680.
[67] Vaccine Design: The Subunit and Adjuvant Approach (eds. Powell & Newman) Plenum Press 1995 (ISBN 0-306-44867-X).
[68] Vaccine Adjuvants: Preparation Methods and Research Protocols (Volume 42 of Methods in Molecular Medicine series). ISBN: 1-59259-083-7. Ed. O'Hagan.
[69] Allison & Byars (1992) Res Immunol 143:519-25.
[70] Hariharan et al. (1995) Cancer Res 55:3486-9.
[71] WO95/11700.
[72] U.S. Pat. No. 6,080,725.
[73] WO2005/097181.
[74] Han et al. (2005) Impact of Vitamin E on Immune Function and Infectious Diseases in the Aged at Nutrition, Immune functions and Health EuroConference, Paris, 9-10 Jun. 2005.
[75] U.S. Pat. No. 6,630,161.
[76] Han et al. (2004) Ann N Y Acad Sci 1031:96-101.
[77] U.S. Pat. No. 6,355,271.
[78] WO00/23105.
[79] U.S. Pat. No. 5,057,540.
[80] WO96/33739.
[81] EP-A-0109942.
[82] WO96/11711.
[83] WO00/07621.
[84] Barr et al. (1998) Advanced Drug Delivery Reviews 32:247-271.
[85] Sjolanderet et al. (1998) Advanced Drug Delivery Reviews 32:321-338.
[86] Pizza et al. (2000) Int J Med Microbiol 290:455-461.
[87] WO95/17211.
[88] WO98/42375.
[89] Singh et al] (2001)J Cont Release 70:267-276.
[90] WO99/27960.
[91] U.S. Pat. No. 6,090,406
[92] U.S. Pat. No. 5,916,588
[93] EP-A-0626169.
[94] Rosenkrands et al. (2005) Infect Immun 73(9):5817-26.
[95] WO99/52549.
[96] WO01/21207.
[97] WO01/21152.
[98] Signorelli & Hadden (2003) Int Immunopharmacol 3(8):1177-86.
[99] Wong et al. (2003) J Clin Pharmacol 43(7):735-42.
[100] US2005/0215517.
[101] Meraldi et al. (2003) Vaccine 21:2485-2491.
[102] Pajak et al. (2003) Vaccine 21:836-842.
[103] WO03/011223.
[104] WO2004/064715.
[105] WO02/072012.
[106] Cooper (1995) Pharm Biotechnol 6:559-80.
[107] Heeg & Zimmerman (2000) Int Arch Allergy Immunol. 121(2):87-97.
[108] Wheeler et al. (2001) International Archives of Allergy and Immunology 126:135-139
[109] He et al. (2000) Clin Diagn Lab Immunol 7(6): 899-903.
[110] Silva et al. (2004) Immunol Cell Biol 82(6):611-6.
[111] Thompson et al. (2005) Journal of Leukocyte Biology 78:1273-1280
[112] Hayden et al. (1998) J Clin Invest 101(3):643-9.
[113] Tassignon et al. (2005) J Immunol Meth 305:188-98.
[114] Myers et al. (1990) pages 145-156 of Cellular and molecular aspects of endotoxin reactions.
[115] Ulrich (2000) Chapter 16 (pages 273-282) of reference 67.
[116] Johnson et al. (1999) J Med Chem 42:4640-9.
[117] Baldrick et al. (2002) Regulatory Toxicol Pharmacol 35:398-413.
[118] U.S. Pat. No. 4,680,338.
[119] U.S. Pat. No. 4,988,815.
[120] WO92/15582.
[121] Stanley (2002) Clin Exp Dermatol 27:571-577.
[122] Wu et al. (2004) Antiviral Res. 64(2):79-83.
[123] Vasilakos et al. (2000) Cell Immunol. 204(1):64-74.
[124] U.S. Pat. Nos. 4,689,338, 4,929,624, 5,238,944, 5,266,575, 5,268,376, 5,346,905, 5,352,784, 5,389,640, 5,395,937, 5,482,936, 5,494,916, 5,525,612, 6,083,505, 6,440,992, 6,627,640, 6,656,938, 6,660,735, 6,660,747, 6,664,260, 6,664,264, 6,664,265, 6,667,312, 6,670,372, 6,677,347, 6,677,348, 6,677,349, 6,683,088, 6,703,402, 6,743,920, 6,800,624, 6,809,203, 6,888,000 and 6,924,293.
[125] Jones (2003) Curr Opin Investig Drugs 4:214-218.
[126] WO2004/060308.
[127] WO2004/064759.
[128] U.S. Pat. No. 6,924,271.
[129] US2005/0070556.
[130] U.S. Pat. No. 5,658,731.
[131] U.S. Pat. No. 5,011,828.
[132] WO2004/87153.
[133] U.S. Pat. No. 6,605,617.
[134] WO02/18383.
[135] WO2004/018455.
[136] WO03/082272.
[137] Dyakonova et al. (2004) Int Immunopharmacol 4(13):1615-23.
[138] FR-2859633.
[139] WO2006/002422.
[140] Johnson et al. (1999) Bioorg Med Chem Lett 9:2273-2278.
[141] Evans et al. (2003) Expert Rev Vaccines 2:219-229.
[142] Andrianov et al. (1998) Biomaterials 19:109-115.
[143] Payne et al. (1998) Adv Drug Delivery Review 31:185-196.
[144] Thompson et al. (2003) Methods in Molecular Medicine 94:255-266.
[145] Kandimalla et al. (2003) Nucleic Acids Research 31:2393-2400.
[146] WO02/26757.
[147] WO99/62923.
[148] Krieg (2003) Nature Medicine 9:831-835.
[149] McCluskie et al. (2002) FEMS Immunology and Medical Microbiology 32:179-185.
[150] WO98/40100.
[151] U.S. Pat. No. 6,207,646.
[152] U.S. Pat. No. 6,239,116.
[153] U.S. Pat. No. 6,429,199.
[154] Kandimalla et al. (2003) Biochemical Society Transactions 31 (part 3):654-658.
[155] Blackwell et al. (2003) J Immunol 170:4061-4068.
[156] Krieg (2002) Trends Immunol 23:64-65.
[157] WO01/95935.
[158] Kandimalla et al. (2003) BBRC 306:948-953.
[159] Bhagat et al. (2003) BBRC 300:853-861.
[160] WO03/035836.
[161] WO01/22972.
[162] Jiao et al. (2004) J Gen Virol 85(Pt 6):1545-53.
[163] Thompson et al. (2005) J Leukoc Biol 78: ‘The low-toxicity versions of LPS, MPL® adjuvant and RC529, are efficient adjuvants for CD4+ T cells’.
[164] UK patent application GB-A-2220211.
[165] WO 94/21292.
[166] WO94/00153.
[167] WO95/17210.
[168] WO96/26741.
[169] WO93/19780.
[170] Gennaro (2000) Remington: The Science and Practice of Pharmacy. 20th edition, ISBN: 0683306472.
[171] Banzhoff (2000) Immunology Letters 71:91-96.
[172] Nony et al. (2001) Vaccine 27:3645-51.
[173] WO2005/089837.
[174] U.S. Pat. No. 6,692,468.
[175] WO00/07647.
[176] WO99/17820.
[177] U.S. Pat. No. 5,971,953.
[178] U.S. Pat. No. 4,060,082.
[179] EP-A-0520618.
[180] WO98/01174.
[181] Potter & Oxford (1979) Br Med Bull 35: 69-75.
[182] Greenbaum et al. (2004) Vaccine 22:2566-77.
[183] Zurbriggen et al. (2003) Expert Rev Vaccines 2:295-304.
[184] Piascik (2003) J Am Pharm Assoc (Wash D.C.). 43:728-30.
[185] Mann et al. (2004) Vaccine 22:2425-9.
[186] Halperin et al. (1979) Am J Public Health 69:1247-50.
[187] Herbert et al. (1979) J Infect Dis 140:234-8.
[188] Chen et al. (2003) Vaccine 21:2830-6.

1.-34. (canceled)

35. A method of raising an immune response in a human comprising administering a composition to the human, wherein the composition comprises a split influenza virus antigen and a squalene-in-water emulsion adjuvant, and wherein the squalene-in-water emulsion adjuvant includes free surfactant in its aqueous phase.

36. The method of claim 35, wherein the split influenza virus antigen is from a H1, H2, H3, H5, H7, or H9 influenza A virus subtype.

37. The method of claim 35, wherein the split influenza virus antigen is derived from an influenza virus strain grown in cell culture, and wherein the composition is free of ovalbumin, ovomucoid, and chicken DNA.

38. The method of claim 37, wherein the composition contains less than 10 ng of host cell DNA.

39. The method of claim 35, wherein the squalene-in-water emulsion adjuvant comprises alpha-tocopherol.

40. The method of claim 35, wherein the squalene-in-water emulsion adjuvant comprises Polysorbate 80 in its aqueous phase.

41. The method of claim 35, wherein the squalene-in-water emulsion adjuvant comprises a 3-O-deacylated monophosphoryl lipid A.

42. The method of claim 35, wherein the squalene-in-water emulsion adjuvant has droplets with a sub-micron diameter.

43. The method of claim 35, wherein the composition includes split influenza virus antigens derived from two influenza A strains and one influenza B strain.

44. The method of claim 35, wherein the composition is a monovalent vaccine against a pandemic influenza virus strain.

45. A method of reducing antigen aggregate in an immunogenic vaccine comprising admixing a split influenza virus antigen with a squalene-in-water emulsion adjuvant to form the immunogenic vaccine, wherein the squalene-in-water emulsion adjuvant includes free surfactant in its aqueous phase.

46. The method of claim 45, wherein the split influenza virus antigen is from a H1, H2, H3, H5, H7, or H9 influenza A virus subtype.

47. The method of claim 45, wherein the split influenza virus antigen is derived from an influenza virus strain grown in cell culture, and wherein the immunogenic vaccine is free of ovalbumin, ovomucoid, and chicken DNA.

48. The method of claim 47, wherein the immunogenic vaccine contains less than 10 ng of host cell DNA.

49. The method of claim 45, wherein the squalene-in-water emulsion adjuvant comprises alpha-tocopherol.

50. The method of claim 45, wherein the squalene-in-water emulsion adjuvant comprises Polysorbate 80 in its aqueous phase.

51. The method of claim 45, wherein the squalene-in-water emulsion adjuvant comprises a 3-O-deacylated monophosphoryl lipid A.

52. The method of claim 45, wherein the squalene-in-water emulsion adjuvant has droplets with a sub-micron diameter.

53. The method of claim 45, wherein the immunogenic vaccine includes split influenza virus antigens derived from two influenza A strains and one influenza B strain.

54. The method of claim 45, wherein the immunogenic vaccine is a monovalent vaccine against a pandemic influenza virus strain.
